(19) United States
(12) Patent Application Publication
(10) Pub. No.: US 2018/0000348 A1
(43) Pub. Date: Jan. 4, 2018

(54) METHOD AND APPARATUS FOR MEDICAL DIAGNOSIS BASED ON THE TISSUE STIFFNESS

(71) Applicant: AUGMENTED MEDICAL INTELLIGENCE LABS, INC., Cambridge, MA (US)

(72) Inventors: Andrew Mina Bishara, Cambridge, MA (US); Marc David Succi, Cambridge, MA (US); Fransiska Putri Wina Hadiwidjana, Surabaya (ID); Elishai Ezra, Jerusalem (IL)

(21) Appl. No.: 15/707,809

(22) Filed: Sep. 18, 2017

Related U.S. Application Data
(63) Continuation-in-part of application No. 13/970,423, filed on Aug. 19, 2013.
(60) Provisional application No. 61/684,629, filed on Aug. 17, 2012.

(51) Int. Cl. A61B 5/00; A61B 5/103

(52) U.S. Cl. CPC A61B 5/0053; A61B 5/6806; A61B 5/103; A61B 2562/0247

(57) ABSTRACT

The present disclosure includes a medical device which can assist in detecting tissue properties. For example, the present disclosure includes a medical device which can detect biological properties associated with the health of a patient and the tissue being examined, as well as cancerous tissue for detecting cancer in a patient. In addition, the medical device of the present disclosure can be used for collecting the data needed to create a predictive model and imaging of the biological tissue being examined.

### REFERENCE TO PRIORITY DOCUMENT

This application is a Continuation-in-Part (CIP) of our co-pending U.S. application Ser. No. 13/970,423, filed Aug. 19, 2013, which application in turn claims the benefit of priority under 35 U.S.C. §119(e) of U.S. Provisional Patent Application Ser. No. 61/684,629, filed Aug. 17, 2012, under 37 C.F.R. §1.78(a). Priority of the filing date is hereby claimed and the full disclosure of the aforementioned application is incorporated herein by reference.

### FIELD

The subject matter described herein relates to embodiments of medical devices and methods for detecting properties of tissue, such as stiffness, elasticity, density, water or electrolyte content, conductivity and optical reflectivity to name a few.

### BACKGROUND

Palpation can be used as part of a physical examination in which a part of a patient's body, such as an organ or area of tissue, is felt by the hands of a healthcare practitioner in order to determine one or more characteristics or properties related to that part of the patient's body. In some cases, palpation is used to detect painful areas (known as tenderness) and to qualify pain felt by the patient. Palpation can also be used for examining breast tissue, such as for detecting cancerous masses in the breast. Palpation can change the physiological properties of the tissue and measuring these changes as a measured perturbation is applied provide insight into the physical properties of both healthy and diseased tissue.

### SUMMARY

Disclosed herein are devices and methods related to embodiments of a medical device for monitoring physiological conditions. An embodiment of the medical device may include a glove body configured to fit over a hand of a user with at least one force sensor secured to at least one fingertip and at least one additional sensor used for detecting biologic conditions secured to at least one additional fingertip of the glove body. In addition, the medical device may include a displacement assessment device (which may be an external sensor such as a light or infrared camera) configured to collect data indicating displacement of at least one fingertip and/or the entire hand as it is pressed against tissue in order to determine the elastic modulus or so-called “stiffness” of the tissue. Additionally, the medical device may include a processor configured to process data obtained by the displacement assessment device to assess the stiffness of the tissue in contact with the at least one force sensor. This processor can be on the glove or allow for wireless sending and receiving of data to a cloud CPU to allow for more advanced data processing including analyses of the multiple real-time changes to the tissue properties (shear strain, stiffness, conductivity, ultrasound picture, tissue perfusion, etc). The analyses uses predictive analytics (specifically deep learning) to build unseen neural networks to model the changes in the data to make future predictions of how the tissue might change with another pertubation. The ultrasonic Two dimensional (2D) images will have a Three (3D) dimensional location in space which can be overlaid with appropriate 3D dimensional position and rotation to make a three dimensional (3D) model of the tissue examined, with the physiologic dynamic properties overlaid. This 3D model will allow for 3D modeling of the tissue to make a physical representation using tools such as 3D printing.

In one embodiment there is provided a medical device comprising: a glove body configured to fit over a hand of a user and at least one force sensor secured to at least one fingertip of the glove body; a displacement assessment device configured to collect data indicating displacement of at least one fingertip as it is pressed against tissue in order to determine the compliance of the tissue; said glove body further comprises one or more electrodes on one or more fingertips of the glove body adapted to make contact with the tissue, and being in electrical connection with a processor and power source; and a processor adapted to process data obtained by the displacement assessment device and the at least one force sensor and the two or more sensors to assess a elasticity and compliance of the tissue and/or biological/electrical properties of the tissue in contact with the at least one force sensor. In such embodiment, the displacement assessment device may comprise a camera or a sensor array of accelerometers or other measurement devices (on or surrounding the glove) for precise 3D mapping of glove position. If desired, the camera may be coupled to the glove body.

In a preferred embodiment the electrodes/sensors are made of a metal, metal oxide, carbon, graphite, conductive polymer, piezoelectric crystal, or combination thereof.

In another embodiment, the displacement assessment device comprises a circumferential pressure sensor array located along at least one fingertip of the glove body and is comprised of a plurality of pressure sensors positioned a defined distance apart in a circumferential arrangement relative to the fingertip. In such embodiment, the pressure sensors may include piezoelectric and resistive transducers. Also, in such embodiment, the processor may be adapted to determine the displacement of the finger against the tissue by evaluating the number of pressure sensors that are in contact with the tissue and the displacement between each pressure sensor.

In another embodiment the pressure sensors may comprise a circumferential pressure sensory array comprised of concentric rings of pressure sensors configured to progress up an edge of the fingertip and positioned a defined distance apart from each other and which at least partially encircle a centrally located force sensor on the fingertip.

In still another embodiment the device may comprise a wrist enclosure configured to contain the processor, which optionally may include a display mounted on the wrist enclosure and configured to display information related to the stiffness of the tissue.

In another embodiment an accelerometer is mounted on the glove body and provide data to the processor to determine the motion of the glove body.

In still yet another embodiment, the assessment device is a camera adapted to record at least a pre-palpation image and a post-palpation image, and/or to precisely 3D map the glove position or location of the glove in space relative to the patient.

Also provided is a medical device comprising: a glove body configured to fit over the hand of a user, said glove having at least two different types of sensors, including a first sensor adapted to collect data indicating a displacement of at least one fingertip when it is pressed against tissue in order to determine a stiffness of the tissue, and a second sensor type adapted to determine another physical or biological property of tissue, secured to two or more fingertips and a thumb of the glove body; and a processor adapted to process data obtained by the first and second sensors for determining physical or biological properties of tissue both in real time and predicatively. In the displacement assessment device is a camera, or a circumferential pressure sensor array. In a preferred embodiment, one or more of the sensors are made of a metal, metal oxide, carbon, graphite, conductive polymer or combination of these. In another preferred embodiment, the displacement assessment device is a circumferential pressure sensor array located along at least one fingertip of the glove body and is comprised of a plurality of pressure sensors positioned a defined distance apart in a circumferential arrangement relative to the fingertip. In such embodiment, the pressure sensors preferably include piezoelectric and resistive transducers, and the processor preferably is adapted to determine a displacement of the finger against the tissue by evaluating the number of pressure sensors that are in contact with the tissue and the displacement between each pressure sensor.

In yet another embodiment, the displacement assessment device is a circumferential pressure sensor array comprised of concentric rings of pressure sensors configured to progress up an edge of the fingertip and positioned a defined distance apart from each other and which at least partially encircle a centrally located force sensor on the fingertip.

In yet another embodiment, the processor is adapted to determine an elastic modulus of the tissue; the processor is contained in a wrist enclosure, and optionally includes a display mounted on the wrist enclosure and adapted to display information related to a stiffness of the tissue, many accelerometers adapted to provide data to the processor to determine a location of the glove body relative to one or more reference points along the body of the patient, is carried on the glove body, and/or a camera is provided and is adapted to record at least a pre-palpation image and a post-palpation image.

An embodiment of a method can include using a medical device for obtaining data characterizing dynamic physiological properties of tissue and may include securing a medical device to a hand of a user. The medical device may include a glove body configured to fit over a hand of a user with at least one force sensor or other sensor) secured to at least one fingertip of the glove body, a displacement assessment device or camera configured to collect data indicating displacement of at least one fingertip as it is pressed against tissue in order to determine the stiffness of the tissue, and a processor configured to process data obtained by the displacement assessment device to assess the stiffness of the tissue in contact with the at least one force sensor. In addition, the method may include placing at least one force sensor against the tissue of a patient and collecting data associated with the force sensor and data associated with displacement assessment device. An important component here are the various maneuvers applied by the users to the patient's tissues are immeasurable and they all add value and information to the device's representation/understanding of the tissue. Maneuvers might include applying force at different angles to the tissue, percussion to measure a change in vibration of the tissue (physicians do this to figure out if there is fluid around the lung or not), squeezing the tissue in a tensile manner, but also in a shear stress manner, which is done during the breast exam. Applying force to skin then releasing quickly to assess how quickly blood refills the capillaries of one's finger or the skin around a mole. Holding the hand in one place and scanning back and forth like using an ultrasound to “see” structures within the heart, or holding the hand in one place to record the sounds of the heart (the valves snapping shut) with a microphone, etc. The device also could be used to measure the tissue properties of a patient's spine and to create a 3D printed model of a patient's spine prior to epidural placement, so that the practitioner could practice placing an epidural in the 3D printed model prior to placing in the patient. Prior imaging that a patient may have undergone as part of other testing (MRI, CT scan, xray) also might be included as part of the device data to ensure an accurate reconstruction of the patient's anatomy and tissue. Additionally, the method may include processing the data and determining properties of the tissue such as mentioned above.

Also provided is a method of using a medical device for obtaining data characterizing physical and/or biological properties of tissue, comprising: providing medical device comprising a glove body configured to fit over a hand of a user said glove having at least two different types of sensors, including a first sensor comprising a displacement assessment device adapted to collect data indicating a displacement of at least one fingertip as it is pressed against tissue in order to determine a stiffness of the tissue, and at least one additional sensor on at least one additional fingertip adapted to be pressed against tissue in order to determine an additional physical or biological property of the tissue, and a processor configured to process data obtained by the displacement assessment device and the at least one additional sensor; placing at least one force sensor and the at least one additional sensor against the tissue of the patient; collecting data associated with the force sensor and the at least one additional sensor; processing the data; and determining a stiffness and or additional property of the tissue.

In one embodiment of the method the displacement assessment device is a camera or is a circumferential or surrounding sensor array. There are a few possibilities for sensing or recording the displacement of the glove body in space. One uses an external camera. Another uses an external surrounding sensor array, e.g. an infrared camera with infrared reflectors on the glove, similar to how infrared cameras and infrared reflectors are used in biomechanics laboratories to monitor walking or running mechanics. Another employs a circumferential set of sensors on the glove that may include many accelerometers that together are able to predict the location of the glove in space with respect to a reference point, using deep or predictive learning. A circumferential sensor array of pressure sensors also potentially could show the orientation of the hand in space, e.g. two fingers out the rest of the fingers clasped etc. And, pointing and pushing of the finger to get the displacement of one finger if the glove body does not move, but the finger moves forward that a circumferential sensor array of pressure sensors could provide a measurement of displacement, and distinguish between hand and finger orientation and overall glove body displacement with respect to the location of the patient body. Preferably the displacement assessment device is a circumferential pressure sensor array located along at least one fingertip of the glove body and is comprised of a plurality of pressure sensors positioned a defined distance apart in a circumferential arrangement relative to the fingertip. In one embodiment the pressure sensors preferably may include piezoelectric and resistive transducers which may be used for examining the inside structure of the tissue (anatomy and fluid movement measuring vessel velocities etc), although piezoelectric crystals also can be used for other types of tissue property discovery and characterization processor preferably determines a displacement of the finger against the tissue by evaluating the number of pressure sensors that are in contact with the tissue and the displacement between each pressure sensor, and wherein the circumferential pressure sensor array is preferably comprised of concentric rings of pressure sensors configured to progress up an edge of the fingertip and positioned a defined distance apart from each other and which at least partially encircle a centrally located force sensor on the fingertip.

In another preferred embodiment, the processor is adapted to calculate elasticity or stiffness of the tissue. As used herein elasticity, Young's modulus, stiffness and mechanical compliance are all used interchangeably. In essence, one can think of the device of the present disclosure as being a “stiffness tensor”, which is a matrix representation of the mechanical compliance of the tissue in six dimensions (include 3 tranlational dimensions and 3 rotational dimensions) and can be applied to any number of points in space. This matrix representation is a characterization of anistropic (materials that have stiffness that varies by the direction in which the stress comes) materials via Hooke's law. In essence, the stiffness tensor may be a 6×6 matrix of basically many spring constants that characterize a point in the tissue and describe how that part of tissue would respond to stresses in various directions and manners.

In yet another embodiment, the medical device further comprises a wrist enclosure configured to contain the processor, wherein the medical device preferably further comprises a display mounted on the wrist enclosure and adapted to display information related to the stiffness of the tissue.

In still yet another embodiment, many accelerometers are mounted on the glove body and provide data to the processor to determine a location of the glove body relative to one or more reference points along the body of the patient.

In yet another embodiment, a camera or surround array of sensors records at least a pre-palpation image and a post-palpation image.

In still yet another embodiment, a processor is adapted to process data obtained by the displacement assessment device with additional sensor types, and to determine physical or biological properties of tissue both in real time and predicatively, and the processed data preferably is used to build a 3-D image.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

These and other aspects will now be described in detail with reference to the following drawings, in which like numerals depict like parts, and wherein:

FIG. 1 shows an embodiment of a medical device configured to fit over a user's hand and detect electrical properties etc of examined tissue.

FIG. 2 shows an example of a collection of data obtained by the medical device including data obtained from a force sensor and vibration sensor.

FIGS. 3A and 3B show an embodiment of the medical device showing LEDs positioned at distal ends of the middle finger, index finger and a photon sensor or camera on the thumb of the glove body and a wrist enclosure.

FIG. 4 shows an embodiment of the medical device including a plurality of miniature sized sensors.

FIGS. 5, 5A and 5B show an embodiment of the medical device including circumferential pressure sensors located along a part of the distal end, or fingertip, of the glove body for determining force (displacement measured by external camera) relative to the examined tissue.

FIG. 6 grammatically illustrates how independent and dependent variables of input are related to output.

FIG. 7 graphically illustrates using prediction of tissue movement for creation of a 3D simulation model.

FIG. 8 shows an example of using an external camera to measure displacement.

FIG. 9 is similar to FIG. 8 and shows an alternative embodiment; and

FIG. 10 is similar to FIG. 8, and illustrates yet another embodiment.

### DETAILED DESCRIPTION

The present disclosure includes a medical device that can assist in detecting tissue properties in situ or in real time. For example, the present disclosure includes a medical device that can detect tissue stiffness tensor, density, conductivity, sound emittance, and optical wavelengths associated with various health conditions. The present invention can be used to both detect and then track changes over a treatment period for health conditions such as cancerous tissue, changes in tissue properties, and even changes in the blood or interstitial fluids, such as oxygen or glucose levels. In addition, the medical device of the present invention can use a technique similar to a clinical breast exam that can be used in places where some medical equipment and training, such as for conducting mammograms, are lacking, such as in developing countries.

Compared to the mammogram, the clinical breast exam can be much less resource intensive with comparable results. For example, clinical breast exams alone can find a substantial proportion of cancers while requiring fewer resources. However, clinical breast exam sensitivities and specificities can be varied, which can be at least partially attributed to differences in physician skill and patient physical characteristics. This can also be applied to other parts of the physical exam such as assessing the size and elasticity of a patient's liver or listening to a patient's heart with a stethoscope or percussing (tapping firmly) the chest to assess for fluid around the lung. This can also be applied to preparing to do a procedure on a patient that is usually done via feel or sight of the patient's tissue, eg arterial line placement, IV placement, epidural catheter placement, peripheral nerve catheter placement, etc

The present medical device can provide cost-effective identification of tissue abnormalities, which may indicate cancerous tissue, such as cancerous nidus in a breast. While breast cancer is a prominent application, the medical device of the present disclosure can also apply to detecting any mass-based cancer near the skin surface, including testicular, prostate and thyroid cancer. In addition, other mass-based physical exam measurements, including pitting edema, sensing percussion based examinations, and determining the rough size of organs close to the surface of the skin (e.g. the liver) can be facilitated by this technology. The medical device of the present disclosure can also provide for quantification of clinical breast exam results.

The glove also would be useful in preparation for procedures on a patient's tissue (i.e., creating a 3D model of the tissue to practice on prior to the actual procedure) and during the procedure in a similar way that ultrasound is useful now for peripheral nerve blocks and central line placements. The technology also would allow for a real-time representation of the tissue while a needle passes through the tissue and a prediction of which tissues a needle might pass through faster than others and an indicator of when the needle is hitting the desired structure versus undesired structures.

In some embodiments, the medical device of the present disclosure can be composed of a fabric-based glove with at least multiple electrodes, a negative and a positive as well as a pressure sensor associated circuitry secured to a part of the fingertips of the glove. The medical device can assist in detecting abnormalities underlying the skin via, as an example, a combination of electrical properties and applied force using sensors on multiple fingers of the medical device.

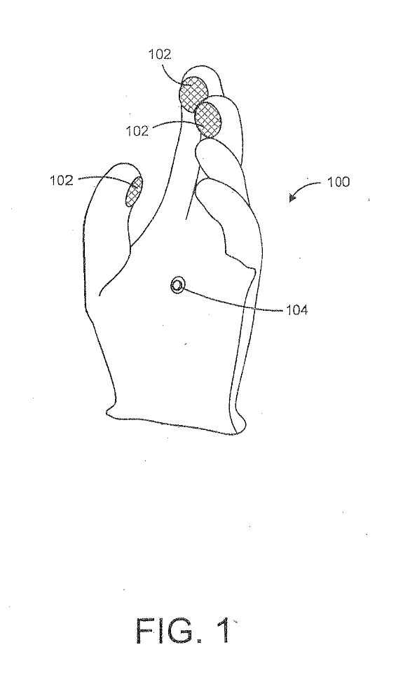

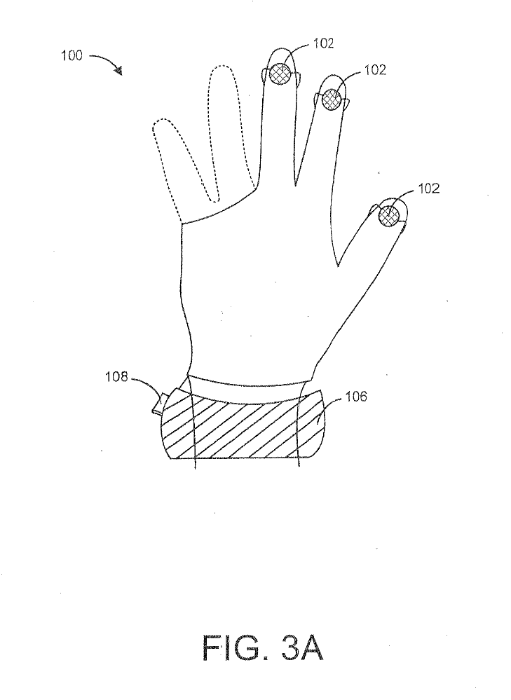

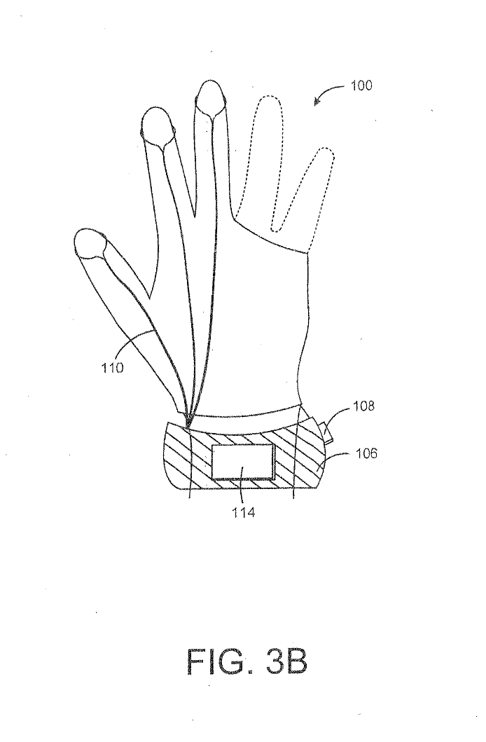

FIG. 1 shows an embodiment of a medical device 100 configured to fit over a hand and detect tissue stiffness, including detecting a variance in stiffness of examined tissue. The medical device 100 includes a glove body having a force sensor 122 positioned at the distal end of a finger, as shown in FIG. 1. In addition, the medical device 100 can include an electrode 126 positioned at the distal end of the thumb and a corresponding opposite electrode 124 positioned at the distal end of another finger. There may additionally be located on the medical device a vibration sensor or camera 104 positioned along the palm of the medical device 100. Additionally, the electrodes, force sensor and vibration sensor may be coupled to a wrist enclosure 106, as shown in FIGS. 3A and 3B, which contains circuitry and electrical components for analyzing and transmitting data to a computer. The wrist enclosure 106 can include a USB connection 108, as shown in FIGS. 3A and 3B, for connecting a USB cable to the wrist enclosure 106 for transmitting data or may be configured for wireless data transfer.

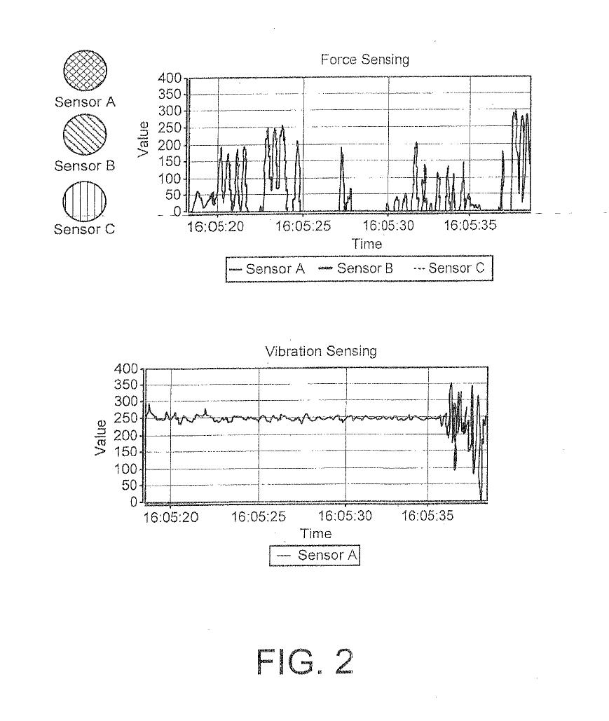

FIG. 2 shows an example of a collection of data obtained by the medical device of the present disclosure including data obtained from a force sensor and vibration sensor. These measurements can be taken from, for example, force sensors 102 positioned on distal ends of the index finger, middle finger and thumb of the glove body of the medical device 100, as shown in FIG. 1. Data obtained from these force sensors 102 can show relative values of force (y axis) and time (x-axis). As shown in FIG. 2, data can also be obtained from a vibration sensor and displayed in graphical form for analysis, such as by a physician. These measurements can assist a user in detecting the stiffness of the examined tissue in order to, for example, detect cancerous tissue.

Some embodiments of the medical device 100 can include sensors that can detect pressure, vibration, acceleration and temperature. In addition, the medical device can include sensors for electronic palpation, galvanic skin conductance sensors, various cameras, including a heat infrared camera, microphones and audible devices, such as buzzers or speakers.

FIGS. 3A and 3B illustrate an embodiment of medical device 100 showing LEDs 132 and 134 of one or more specific wavelengths positioned at distal ends of the middle finger and index finger, a camera or photon sensor 130 is positioned on the distal end of the thumb of the glove body of the medical device 100. In addition, FIGS. 3A and 3B show at least a part of the circuitry or wiring 110 required for the functioning of the LEDs and sensors and for the transmission of the sensed data to the wrist enclosure 106 for storage and further processing. As shown in FIG. 3B, the wires 110 extend between the LEDs and sensors and the wrist enclosure 106 and can run along one side of the glove body, such as the back side of the glove and hand.

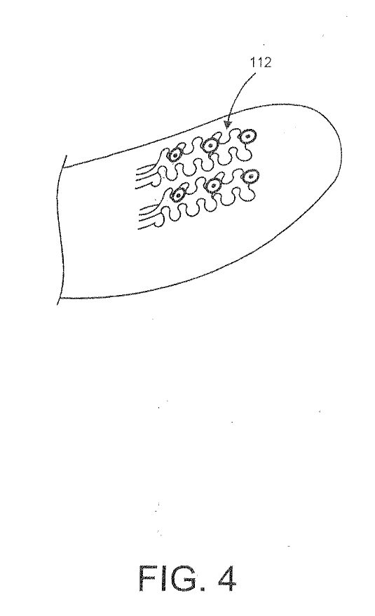

FIG. 4 shows an embodiment of medical device 100 including a plurality of miniature sized sensors 112. The miniature sized sensors 112 can be used to assist in detecting tissue stiffness and cancerous tissue in the patient and can be distally positioned along a finger covering of the glove body.

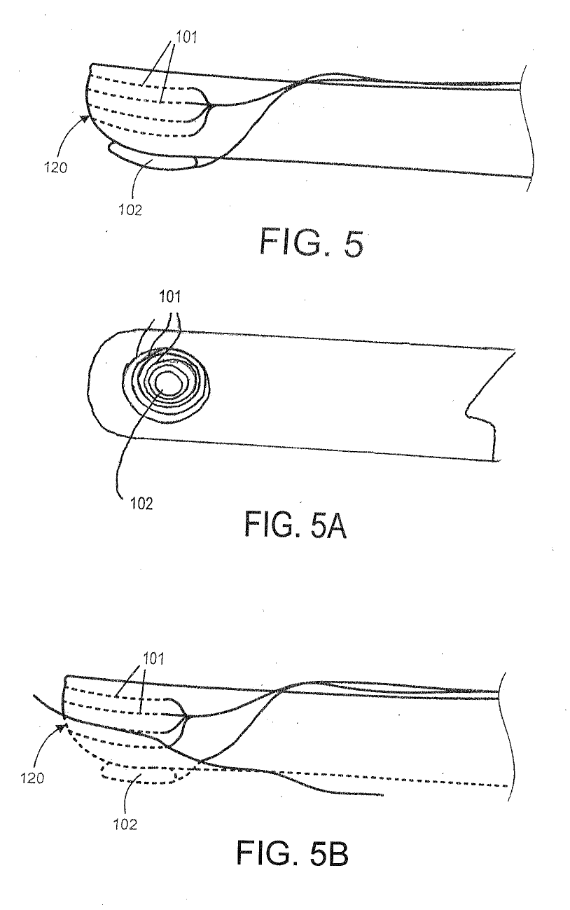

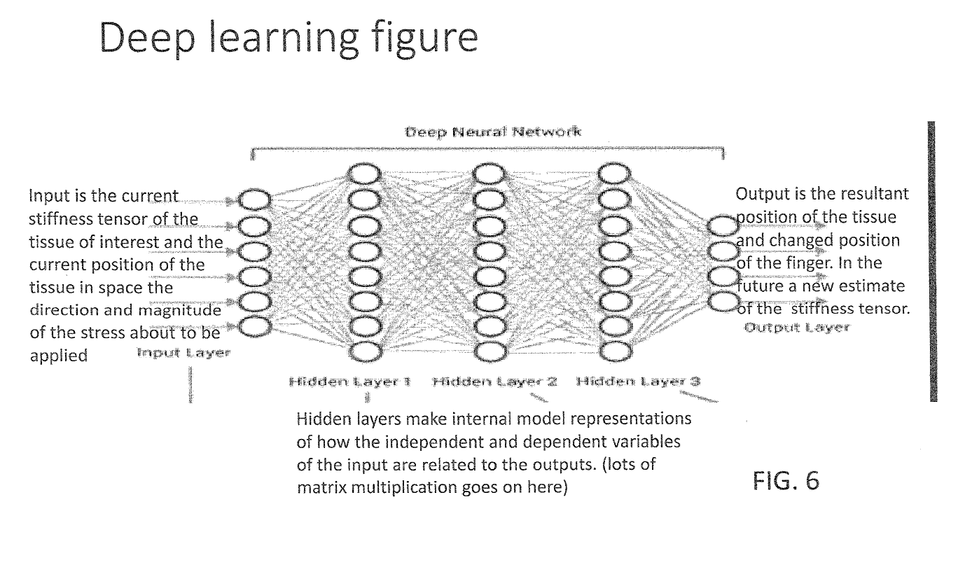

FIGS. 5, 5A and 5B show an embodiment of the medical device including circumferential pressure sensors 120 located along a part of the distal end, or fingertip, of the glove body. The circumferential pressure sensors 120 can be comprised of one or more pressure sensors positioned a defined distance apart from each other in a circumferential arrangement around a distal end of the glove, or finger. The circumferential pressure sensors 120 can be arranged such that as the user presses the glove, or finger, into the body of a patient, as shown in FIG. 6, the more pressure sensors come into contact with the patient due to the user's finger becoming impressed into the patient's body. Therefore, as the user's finger becomes increasingly impressed into the body of the patient, the circumferential pressure sensors 120 will be able to detect the amount of displacement the user's finger has made into the body of the patient by evaluating the number of circumferential pressure sensors 120 contacting the body of the patient and factoring in the displacement between the circumferential pressure sensors 120. In addition, the medical device can include one or more processors for computing and evaluating the sensed data, including from the circumferential pressure sensors 120.

In at least some embodiments of the medical device, the data obtained from the force sensors 102 can be computed and evaluated in conjunction with the electrodes and conductivity or impedance of the tissue being compressed, or impressed, such as described above using circumferential pressure sensors 120, in order to detect the presence or absence of tissue masses under the skin and any changes that occur during compression. In particular, medical device 100 can use such sensed data to detect breast cancer, testicular cancer, etc., and masses such as enlarged lymph nodes.

Some advantages of the medical device include the ability to maintain dexterity of the user, such as a physician or nurse, while still collecting data relating to the tissue being examined, such as tissue stiffness and/or to obtain additional data and guidance while performing a procedure. For example, the glove material of the medical device 100 can include thin spandex, which can facilitate dexterity. In addition, the wrist enclosure 106 can include a plastic housing that can be easily mounted to the operator's wrist and that can contain the necessary power sources, circuits, electronics, processors, user inputs and device outputs of the medical device 100. In the case of sterile procedures the glove can be worn under a sterile glove, which may impair some of the electrical properties or the glove will be prepared with a sterile covering that does not impair sensor data collection or as is the cases with an IV placement the provider will not touch the sterile parts of the catheter or skin during placement.

For example, during a breast exam, a user wearing the medical device 100 can depress the skin of a patient with one or more fingertips, which creates a displacement between the starting position of the finger (i.e., the fingertip placed against the skin of the patient prior to depressing the fingertip against the skin of the patient) and the fully depressed finger. With the assistance of the present medical device 100, a user can perform palpation while the medical device senses the displacement of the one or more fingers into the body or skin of the patient and the amount of force applied against the body or skin of the patient. The medical device can then calculate the associated tissue stiffness using the sensed force from the force sensors 102 and displacement measurements from the circumferential pressure sensors 120. This can also be applied to procedures.

In some embodiments, the force sensor is centered in the middle of the fingertip, with concentric rings 101 of pressure transducers, such a piezoelectric and peizoresistive fabric, and positioned a defined distance apart from each other progressing up the edge of the fingertip, and at least partially encircle a centrally located force sensor 102 on the fingertip, such as is shown in FIGS. 5 and 6. In addition, the rings are placed at known distances from the bottom part of the force sensor 102 located on the fingertip, which can allow a combination of force sensing while determining the depth of tissue palpation via detecting enveloping tissue along the edges of the fingertip.

As mentioned previously, Young's modulus, also known as tensile or elastic modulus, is the measure of the stiffness of an elastic material. In this application, it can be used to characterize the stiffness of abnormal and potentially cancerous tissue. Because cancerous tissue masses will typically be stiffer than surrounding normal tissue, detecting this change in tissue stiffness can be used to alert the operator of a possible abnormal mass, which can indicate malignancy. In at least some instances, cancerous tissues can be as much as seven times as stiff as normal tissues. Also in the case of an epidural placement, the provider is attempting to find the spaces between bones with a needle and uses palpation to find that space and therefore a digital representation of the tissue stiffnesses in a 3D model is useful for predicting optimal location for entering the skin with the needle and for predicting the optimal trajectory of the needle. Also during placement this technology would aid in realizing if there is deviation from the predefined optimal trajectory of the needle for epidural placement (loss of resistances as the needle passes through ligamentum flavum and into the epidural space). Loss of resistance would also be able to be quantified with the glove (which is the sudden loss of stiffness on the back of the syringe as the user is applying pressure to the syringe plunger because the tip of the needle has passed through ligamentum flavum and into the epidural space.

Equation 1, when used in conjunction with the sensed data collected by the medical device 100, can be used to determine Young's modulus using Equation 1 as detailed below, or Hooke's law using Equation 2, as also detailed below. Referring to Equation 1, E represents Young's modulus, F is the force exerted on an object under tension, A₀ is the original cross-sectional area through which the force is applied, ΔL is the amount by which the length of the object changes, L₀ is the original length of the object.

$$E ≡ \frac{tensile stress}{tensile strain} = \frac{σ}{ɛ} = \frac{F / {A}_{0}}{Δ L / {L}_{0}} = \frac{{FL}_{0}}{{A}_{0} Δ L} \qquad Equation 1$$

The calculated Young's modulus differs based on whether the tissue is cancerous or not. The present medical device 100 is able to calculate the Young's modulus by first determining F based on the applied force of the user, such as from sensed data obtained from the force sensors 102 along the distal end of the glove. In addition, the area of the force sensor 102 on the fingertip is variable A₀, the original cross-sectional area through which the force is applied, which may also be equivalent to the area of the fingertip of the glove. One might also use the tip of the finger to feel the radial artery pulse as an anesthesiologist dose when placing an arterial catheter or drawing a blood gas. The maping of the location of the artery and which areas of the artery has the best blood flow is exteremely useful in placement of the arterial catheter and the optimal place and trajectory to stick the needle. This is a real-time change in the stiffness of the tissue that can be sense via the glove.

In order to obtain ΔL and L₀, it is necessary to determine how far the fingertip moves (ΔL), as well as the starting length of the material compressed (L₀,). In some embodiments, this calculation can be determined with the assistance of an external camera in order to obtain two images of the tissue. For example, the camera 104 shown in FIG. 1 can record at least a pre-palpation image and a post-palpation image, which can be used to determine the displacement of the finger against the tissue.

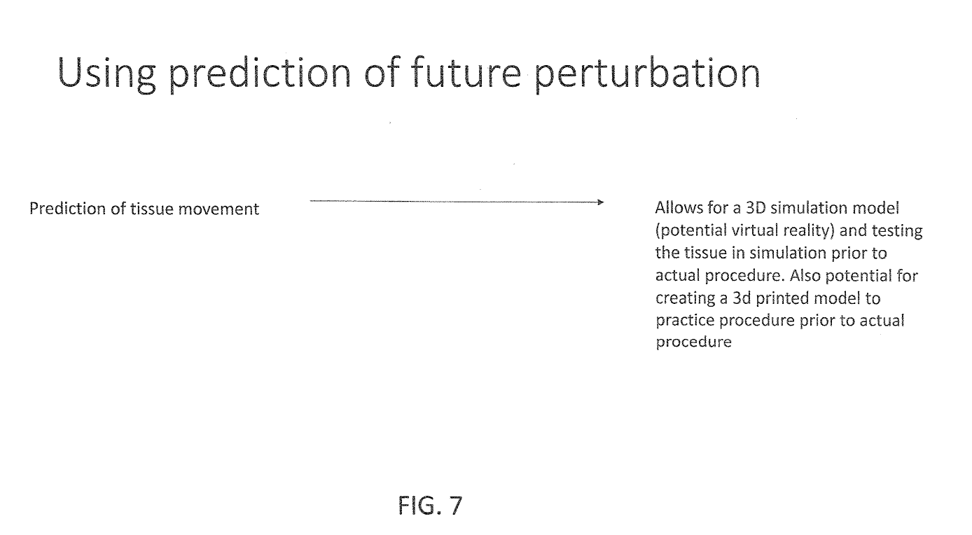

Predictive models also can be obtained by logging data derived from pressure applied at differing angles to the same tissue area via the finger. A three-dimensional matrix of the moduli can be established and used to make predictions of the Young's modulus in an area of tissue recently pressed but at a new angle. These predictions can be created using a deep neural net and are of use to make low cost 3D images of conditions such as a tumor and or other tissue properties in real time as the patient is being examined. See FIGS. 6 and 7.

Analogously, Hooke's law, Equation 2, reproduced below, can be used in conjunction with the sensed data collected and mapped as follows, wherein “ε” represents the strain tensor, “σ” represents the stress tensor, and “C” is the stiffnesstensor or elasticity tensor.

$$\begin{matrix}Hooke ' s law \text{:} [ σ ] = [ C ] [ ɛ ] or {σ}_{i} = {C}_{ij} {ɛ}_{j} . & ( Equation 2 ) \\ [ σ ] = [ \begin{matrix}{σ}_{11} \\ {σ}_{22} \\ {σ}_{33} \\ {σ}_{23} \\ {σ}_{13} \\ {σ}_{12}\end{matrix} ] ≡ [ \begin{matrix}{σ}_{1} \\ {σ}_{2} \\ {σ}_{3} \\ {σ}_{4} \\ {σ}_{5} \\ {σ}_{6}\end{matrix} ] ; [ ɛ ] = [ \begin{matrix}{ɛ}_{11} \\ {ɛ}_{22} \\ {ɛ}_{33} \\ 2 {ɛ}_{23} \\ 2 {ɛ}_{13} \\ 2 {ɛ}_{12}\end{matrix} ] ≡ [ \begin{matrix}{ɛ}_{1} \\ {ɛ}_{2} \\ {ɛ}_{3} \\ {ɛ}_{4} \\ {ɛ}_{5} \\ {ɛ}_{6}\end{matrix} ] \\ Then the stiffness tensor ( c ) can be expressed as \\ [ c ] = [ \begin{matrix}{c}_{1111} & {c}_{1122} & {c}_{1133} & {c}_{1123} & {c}_{1131} & {c}_{1112} \\ {c}_{2211} & {c}_{2222} & {c}_{2233} & {c}_{2223} & {c}_{2231} & {c}_{2212} \\ {c}_{3311} & {c}_{3322} & {c}_{3333} & {c}_{3323} & {c}_{3331} & {c}_{3312} \\ {c}_{2311} & {c}_{2322} & {c}_{2333} & {c}_{2323} & {c}_{2331} & {c}_{2312} \\ {c}_{3111} & {c}_{3122} & {c}_{3133} & {c}_{3123} & {c}_{3131} & {c}_{3112} \\ {c}_{1211} & {c}_{1222} & {c}_{1233} & {c}_{1223} & {c}_{1231} & {c}_{1212}\end{matrix} ] ≡ [ \begin{matrix}{C}_{11} & {C}_{12} & {C}_{13} & {C}_{14} & {C}_{15} & {C}_{16} \\ {C}_{12} & {C}_{22} & {C}_{23} & {C}_{24} & {C}_{25} & {C}_{26} \\ {C}_{13} & {C}_{23} & {C}_{33} & {C}_{34} & {C}_{35} & {C}_{36} \\ {C}_{14} & {C}_{24} & {C}_{34} & {C}_{44} & {C}_{45} & {C}_{46} \\ {C}_{15} & {C}_{25} & {C}_{35} & {C}_{45} & {C}_{55} & {C}_{56} \\ {C}_{16} & {C}_{26} & {C}_{36} & {C}_{46} & {C}_{56} & {C}_{66}\end{matrix} ] & \end{matrix}$$

Alternatively, camera 104 can be mounted on an examining table and can record all operations involving the glove of the medical device, including the motion and displacement associated with palpation. The recorded displacements can then be used to calculate the stiffness, or elastic modulus, of the patient's tissue. In addition, the medical device 100 can include a feedback signal from the camera, which can indicate if the camera's view is being obscured in order to assure correct function of the device, such as capturing the impression of the finger into the examined tissue.

In some embodiments, the medical device 100 can be configured to determine tissue stiffness as well as obtain sensed data from other secondary sensors, including one or more of a thermometer or accelerometer. For example, a thermometer can sense the temperature of the skin being palpated, which may indicate an underlying pathological process or condition.

In addition, many accelerometers mounted on the palmar aspect of the glove of the medical device 100 can allow the medical device 100 to self-locate the glove body relative to the patient body, such as relative to one or more reference points, such as the bellybutton and bilateral axilla. This method requires calibration of the medical device 100 prior to use, and may be able to determine not only the tissue stiffness as previously described, but a rough sense of where the glove body is located relative to the patient's body. Also, the array of the pressure sensors on the glove that can tell how the fingers of the glove would have moved with respect to one another.

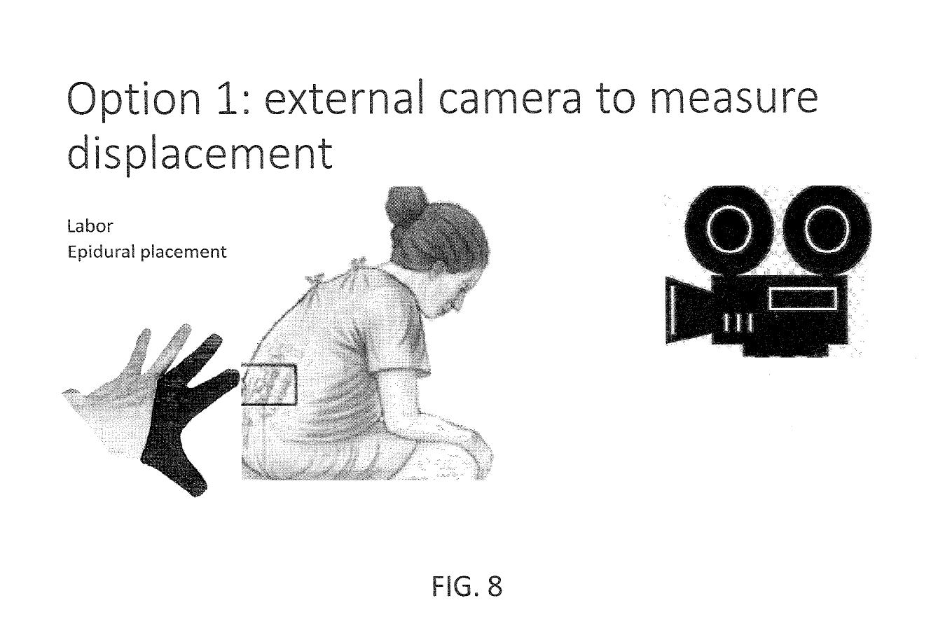

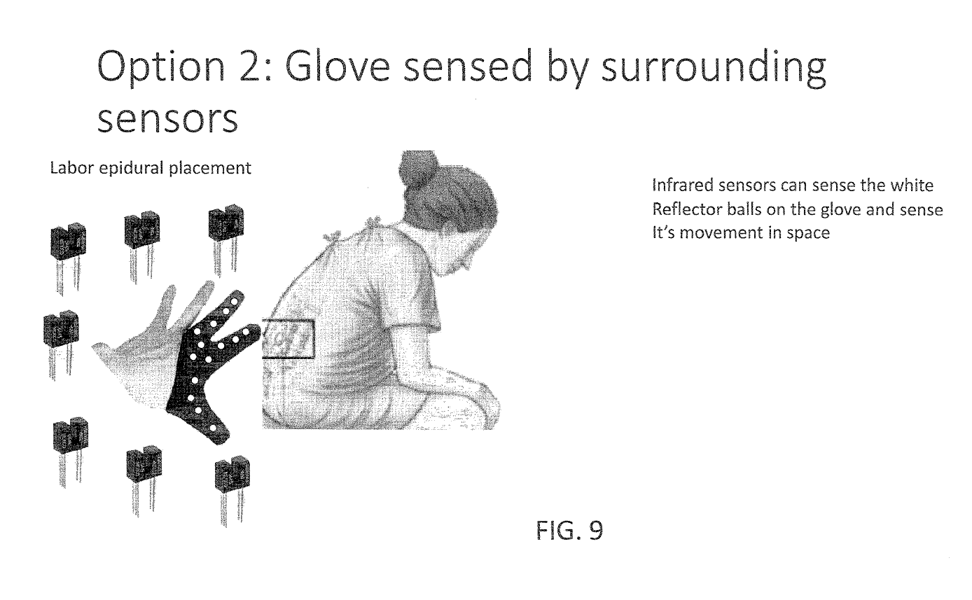

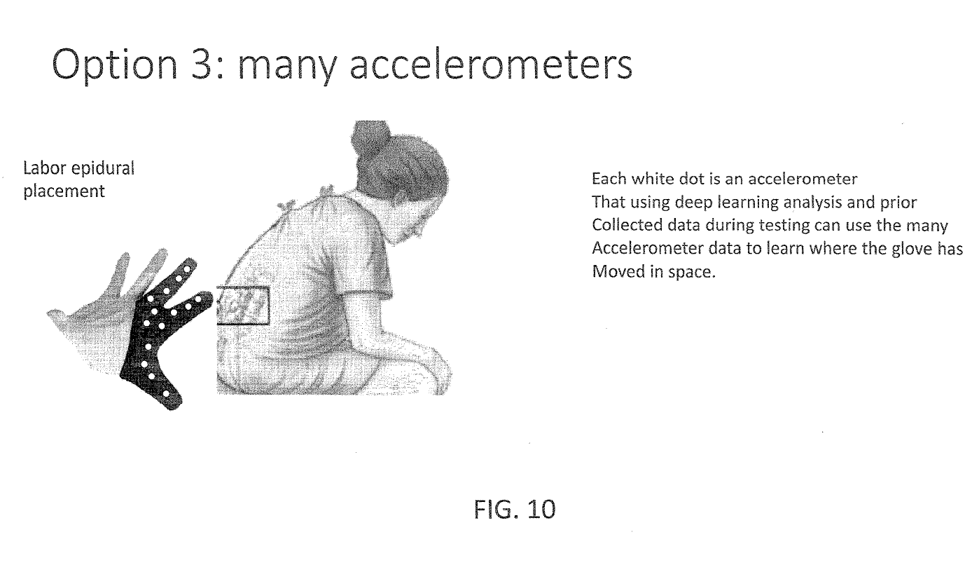

In some embodiments of the medical device 100, an LCD screen can be mounted on the wrist enclosure 106 and can provide some user feedback related to tissue stiffness. The user feedback related to tissue stiffness can be provided on either the LCD screen or on a computer after having connected the wrist enclosure 106 to a computer and transferred the data stored on the wrist enclosure 106. Yet other embodiments are shown in FIGS. 8-10.

Although a few specific embodiments have been described in detail above, other modifications consistent with the spirit of this disclosure are contemplated.

1. A medical device comprising:
a glove body configured to fit over a hand of a user and at least one force sensor secured to at least one fingertip of the glove body on an exterior surface of the glove body;
a displacement assessment sensor configured to collect data indicating displacement of the at least one fingertip as the at least one fingertip is pressed against the tissue of a patient wherein the displacement assessment sensor comprises a circumferential pressure sensor array located along the at least one fingertip of the glove body and comprised of a plurality of pressure sensors positioned a defined distance apart in a circumferential arrangement on the at least one fingertip of the glove, such that in use as the at least one fingertip is pressed against the tissue of a patient, more pressure sensors come into contact with the body of the patient indicative of an amount of the displacement;
a processor configured to process data indicating a displacement of at least one fingertip as the at least one fingertip is pressed against the tissue of the patient obtained by the displacement assessment sensor and the at least one force sensor to determine an elastic modulus of the tissue, and
a camera coupled to the glove body for capturing an image of the fingertip pressed against the tissue.

2. The medical device of claim 1, wherein the pressure sensors include at least one of:
piezoelectric transducers and resistive transducers.

3. The medical device of claim 1, wherein the processor determines the displacement of the at least one fingertip against the tissue by evaluating a number of pressure sensors that are in contact with the tissue and a displacement between each pressure sensor.

4. The medical device of claim 1, wherein the circumferential pressure sensor array is comprised of concentric rings of pressure sensors progressing up an edge of the fingertip and positioned the defined distance apart from each other and which at least partially encircle the at least one force sensor which is centrally located on the fingertip.

5. The medical device of claim 1, further comprising a wrist enclosure configured to contain the processor.

6. The medical device of claim 5, further including a display mounted on the wrist enclosure and configured to display information related to the elastic modulus of the tissue.

7. The medical device of claim 1, wherein an accelerometer is mounted on the glove body and provides data to the processor to determine a location of the glove body relative to one or more reference points along the body of the patient.

8. The medical device of claim 1, wherein the data collected comprises at least a pre-palpation image and a post-palpation image.

9. A method of using a medical device for obtaining data characterizing an elastic modulus E of tissue of a body of a patient, comprising:
providing a medical device, wherein the medical device includes a glove body configured to fit over a hand of the user and having at least one force sensor secured to at least one fingertip of the glove body on an exterior surface of the glove body, a first displacement assessment sensor configured to collect a first quantity of data indicating a displacement of the at least one fingertip when pressed against the tissue of the body of the patient, wherein the displacement assessment sensor is a circumferential pressure sensor array located along the at least one fingertip of the glove body and comprised of a plurality of pressure sensors positioned a defined distance apart in a circumferential arrangement relative to the at least one fingertip of the glove body, such that in use as the at least one fingertip is pressed against the tisse of the body of the patient, more pressure sensors come into contact with the body of the patient indicative of an amount of the displacement, and a camera coupled to the device for capturing an image of the fingertip pressed against the tissue;
sequentially placing the at least one fingertip and the force sensor against the tissue at a first and a second location of a patient;
collecting images of the fingertip pressed against the tissue of the patient, and collecting the first and the second quantity of data associated respectively with the force sensor and the first displacement assessment sensor;
processing the first and second quantities of data; and
calculating the elastic modulus c of the tissue based on the processed first and second quantities of data.

10. The method of claim 9, wherein the pressure sensors include at least one of: piezoelectric transducers and resistive transducers.

11. The method of claim 9, wherein the processor determines the displacement of the at least one fingertip against the tissue by evaluating a number or pressure sensors that are in contact with the tissue and a displacement between each pressure sensor.

12. The method of claim 9, wherein the circumferential pressure sensor array is comprised of concentric rings of pressure sensors progressing up an edge of the fingertip and positioned the defined distance apart from each other and which at least partially encircle the at least one force sensor which is centrally located on the fingertip.

13. The method of claim 9, wherein the medical device further comprises a wrist enclosure configured to contain the processor.

14. The method of claim 13, wherein the medical device further comprises a display mounted on the wrist enclosure and configured to display information related to the elastic modulus of the tissue.

15. The method of claim 9, wherein an accelerometer is mounted on the glove body and provides data to the processor to determine a location of the glove body relative to one or more reference points along the body of the patient.

16. The method of claim 9, wherein the camera records at least a pre-palpation image and a post-palpation image.

17. The medical device of claim 1, further including a sensor adapted to sense one or more of pressure, vibration, acceleration, temperature and galvanic skin conductance.

18. The method of claim 9, wherein the device further includes a sensor adapted to sense one or more of pressure, vibration, acceleration, temperature and galvanic skin conductance.
